(19) United States
(12) Patent Application Publication
(10) Pub. No.: US 2020/0345884 A1
(43) Pub. Date: Nov. 5, 2020

(54) SYSTEM AND METHOD FOR REMOTE CONTROLLING AND MAINTENANCE OF A DEVICE FOR SPREADING ODORS

(71) Applicant: Whitescent Ltd., Netanya (IL)

(72) Inventor: Lior Thau, Kfar Vitkin (IL)

(73) Assignee: Whitescent Ltd., Netanya (IL)

(21) Appl. No.: 16/439,746

(22) Filed: Jun. 13, 2019

(30) Foreign Application Priority Data
Apr. 30, 2019 (IL) 266350

(51) Int. Cl. A61L 9/14; G05B 15/02

(52) U.S. Cl. CPC A61L 9/14; A61L 2209/133; A61L 2209/111; G05B 15/02

(57) ABSTRACT

A device for spreading odors is disclosed. Such a device may include: one or more mist sensors; and two or more liquid-odor providing units. Each liquid-odor providing unit may include one or more replaceable tanks for holding the liquid-odor; one or more pumps for pumping the liquid-odor from the tank; and a ventilation unit for spreading the pumped liquid-odor.

### CROSS REFERENCE TO RELATED APPLICATIONS

This application claims the benefit of Israeli Patent Application Serial No. 266350, filed on Apr. 30, 2019 and entitled SYSTEM AND METHOD FOR REMOTE CONTROLLING AND MAINTENANCE OF A DEVICE FOR SPREADING ODORS, which is incorporated in its entirety herein by reference.

### FIELD OF THE INVENTION

The present invention generally relates to remote controlling of a device for spreading odors. More particularly, the present invention relates to systems and methods for remote controlling and maintenance of a device for spreading odors.

### BACKGROUND OF THE INVENTION

Industrial devices of spreading odors, also known as “industrial scent diffusers,” are devices that are capable of spreading gases and/or aerosols containing aromatic materials in large facilities, such as, shopping malls, hospitals, offices, hotels lobbies and the like. In some cases, the scent diffusers are included in the air-conditioning and ventilation systems of the facilities. Such a device includes: a replaceable cartridge (e.g., tank) containing the odor material, usually in a liquid phase, a pump for pumping the odor material from the cartridge and a ventilation system configured to convert (e.g., diffuse) the odor material into gas or aerosol and spreading the gas or aerosol in the facility (or the ventilation/air conditioning system of the facility).

Most of the currently used industrial scent diffusers are standalone devices that require in premises manual maintenance. Some of the devices include sensors that can indicate a shortage in the amount of odor material in the cartridge or a reduction of the amount of gas/aerosol provided by the device. These sensors may send an alert to a service-provider that the cartridge needs to be replaced or that a technical problem occurred in other components of the device (e.g., pumps, fans, sensors, etc.). In such a case the service-provider will send a technician to solve the problem. Some industrial scent diffusers systems include a spare cartridge and may allow automatic switching between cartridges if the amount of odor material in one cartridge drops below a threshold level. However, such systems lack the ability to remotely detect and solve other technical problems in other components of the industrial scent diffusers.

Accordingly, there is a need for a system that can remotely detect and solve technical problems that may occur in industrial scent diffusers.

### SUMMARY OF THE INVENTION

Some aspects of the invention may be related to a device for spreading odors. Such a device may include: one or more mist sensors; and two or more liquid-odor providing units. In some embodiments, each liquid-odor providing unit may include one or more replaceable tanks for holding the liquid-odor; one or more pumps for pumping the liquid-odor from the tank; and a ventilation unit for spreading the pumped liquid-odor.

In some embodiments, the device may further include two or more mist sensors, each being associated with a different liquid-odor providing unit. In some embodiments, each mist sensor may be located in proximity to an exit of the odor-mist.

In some embodiments, the device may further include: a communication unit; and a processor configured to: receive signal indicating a reduction in the amount of odor-mist provided by the device. In some embodiments, the processor may further be configured to: send the received signal to an external computing device, via the communication unit.

Some aspects of the invention may be related to a method of remote controlling and maintenance of a device for spreading odors. Embodiments of the method may include remotely connecting a device, the device may include one or more mist sensors; and two or more liquid-odor providing units; and receiving from the one or more sensors a signal indicating a reduction in the amount of odor-mist provided by the device. In some embodiments, each liquid-odor providing unit may include one or more replaceable tanks for holding the liquid-odor; one or more pumps for pumping the liquid-odor from the tank; and a ventilation unit for spreading the pumped liquid-odor. Embodiments of the method may further include remotely deactivating a first liquid-odor providing unit and activating a second liquid-odor providing unit when the reduction in the amount of odor-mist is above a predetermined threshold, conducted by shutting down the one or more pumps and the ventilation unit of the first liquid-odor providing unit and starting the operation of the one or more pumps and the ventilation unit of the second liquid-odor providing unit.

Embodiments of the method may further include: receiving an indication in change in the amount of current consumed by at least one of: the ventilation unit and the one or more pumps; and remotely deactivating a first liquid-odor providing unit and activating a second liquid-odor providing unit also based on the received indication.

In some embodiments, each liquid-odor providing unit may be associated with a different sensor and deactivating between the first liquid-odor providing unit and activating the second liquid-odor providing unit further includes shutting-down the senor of the first liquid-odor providing unit and operating the sensor of the second liquid-odor providing unit.

Embodiments of the method may further include: receiving information related to the total operation time of the device since a replacement of a first full tank; calculating an amount of liquid-odor consumed during the total operation time; and remotely deactivating the first liquid-odor providing unit and activating the second liquid-odor providing unit.

Embodiments of the method may further include sending a request for a service call.

Some aspects of the invention may be related to a system for remote controlling and maintenance of devices for spreading odors. The system may include: at least one controller; and a communication unit configured to communicate between the at least one controller and one or more devices for spreading odors. In some embodiments, each of the devices may include: one or more mist sensors; and two or more liquid-odor providing units. In some embodiments, each liquid-odor providing unit may include: one or more replaceable tanks for holding the liquid-odor; one or more pumps for pumping the liquid-odor from the tank; and a ventilation unit for spreading the pumped liquid-odor. In some embodiments, the controller may be configured to: receive from the one or more sensors a signal indicating a reduction in the amount of odor-mist provided by the device; and remotely deactivate a first liquid-odor providing unit and activate a second liquid-odor providing unit when the reduction in the amount of odor-mist is above a predetermined threshold, conducted by shutting down the one or more pumps and the ventilation unit of the first liquid-odor providing unit and starting the operation of the one or more pumps and the ventilation unit of the second liquid-odor providing unit.

Some aspects of the invention may be related to a method of remote controlling and maintenance of a device for spreading odors including: remotely connecting a device comprising: two or more liquid-odor providing units. In some embodiments, each liquid-odor providing unit may include: one or more replaceable tanks for holding the liquid-odor; one or more pumps for pumping the liquid-odor from the tank; and a ventilation unit for spreading the pumped liquid-odor. In some embodiments, the method may include identifying a change in the amount of current consumed by at least one of: the ventilation unit and the one or more pumps; and remotely deactivating a first liquid-odor providing unit and activating a second liquid-odor providing unit based on the identified change in the consumed current, conducted by shutting down the one or more pumps and the ventilation unit of the first liquid-odor providing unit and starting the operation of the one or more pumps and the ventilation unit of the second liquid-odor providing unit.

Embodiments of the method may include: identifying a change in the amount of current consumed may include one of: identifying an increase in the consumed current above a first predetermined threshold value; and identifying a decrease in the consumed current below a second predetermined threshold value.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows a block diagram of a computing device, according to some embodiments of the invention;

FIG. 2 shows a block diagram of a system for remote controlling and maintenance of devices for spreading odors, according to some embodiments of the invention;

FIG. 3A-3C are illustrations of devices for spreading odors according to some embodiments of the invention;

FIG. 4 is a flowchart of a method of remote controlling and maintenance of devices for spreading odors, according to some embodiments of the invention; and

FIG. 5 is a flowchart of a method of remote controlling and maintenance of devices for spreading odors, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF EMBODIMENTS OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing”, “computing”, “calculating”, “determining”, “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

Aspects of the invention may be related to a system and method for remote controlling and maintenance of devices for spreading odors also known in the art as “scent diffusers”. The system and method according to embodiments of the invention may allow to remotely detect a reduction in the amount of odor-mist provided by each device regardless of the reason (e.g., running out of odor material, malfunctioning of one of the devices components and the like). The system and method may further allow to remotely overcome the reduction in the amount of the odor-mist by remotely providing a solution to several problems that may occur in the device. The system and method according to embodiments of the invention may allow to remotely deactivating (by providing a computer command) a first liquid-odor providing unit included in the device and activating a second liquid-odor providing unit included in the device. Each one of the liquid-odor providing units may include: a separate replaceable cartridge (e.g., a tank for holding the liquid-odor), a separate pump and a separate ventilation unit for spreading a pumped liquid-odor. In some embodiments, each liquid-odor providing unit may further be associated with or may include a separate mist-sensor.

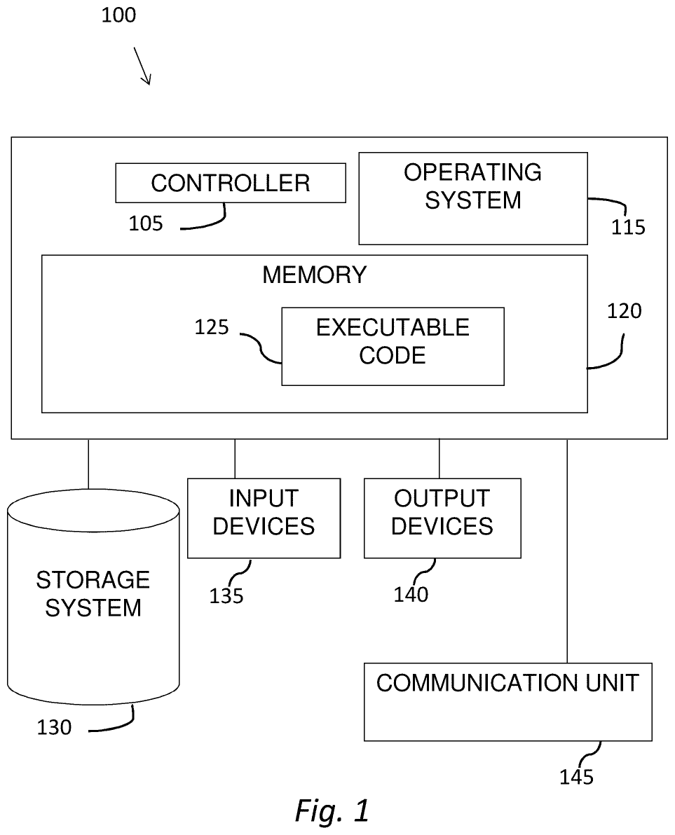

Reference is made to FIG. 1, which is a schematic block diagram of an example of a computing device, according to some embodiments of the invention. Computing device 100 may be or may be included in a system for remote controlling and maintenance of devices for spreading odors. Computing device 100 may include a controller or processor 105 (e.g., a central processing unit processor (CPU), a graphics processing unit (GPU), a chip or any suitable computing or computational device), an operating system 115, memory 120, executable code 125, storage 130, input devices 135 (e.g. a keyboard or touchscreen), and output devices 140 (e.g., a display), a communication unit 145 (e.g., a cellular transmitter or modem, a Wi-Fi communication unit, or the like) for communicating with remote devices via a communication network, such as, for example, the Internet. Controller 105 may be configured to execute program code to perform operations described herein. The system described herein may include one or more computing device(s) 100.

Operating system 115 may be or may include any code segment (e.g., one similar to executable code 125 described herein) designed and/or configured to perform tasks involving coordinating, scheduling, arbitrating, supervising, controlling or otherwise managing operation of computing device 100, for example, scheduling execution of software programs or enabling software programs or other modules or units to communicate.

Memory 120 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 120 may be or may include a plurality of, possibly different memory units. Memory 120 may be a computer or processor non-transitory readable medium, or a computer non-transitory storage medium, e.g., a RAM.

Executable code 125 may be any executable code, e.g., an application, a program, a process, task or script. Executable code 125 may be executed by controller 105 possibly under control of operating system 115. For example, executable code 125 may be a software application that performs methods as further described herein, for example, methods of remote controlling and maintenance of devices for spreading odors. Although, for the sake of clarity, a single item of executable code 125 is shown in FIG. 1, a system according to embodiments of the invention may include a plurality of executable code segments similar to executable code 125 that may be stored into memory 120 and cause controller 105 to carry out methods described herein.

Storage 130 may be or may include, for example, a hard disk drive, a universal serial bus (USB) device or other suitable removable and/or fixed storage unit. In some embodiments, some of the components shown in FIG. 1 may be omitted. For example, memory 120 may be a non-volatile memory having the storage capacity of storage 130. Accordingly, although shown as a separate component, storage 130 may be embedded or included in memory 120.

In some embodiments, computing device 100 may be hosted in a cloud-based processing service, such that both professor 105 and memory 120 may be located on the cloud. In some embodiments, storage 130 may be host in cloud-based database and may communicate with computing device 100 via the interne.

Input devices 135 may be or may include a keyboard, a touch screen or pad, one or more sensors or any other or additional suitable input device. Any suitable number of input devices 135 may be operatively connected to computing device 100. Output devices 140 may include one or more displays or monitors and/or any other suitable output devices. Any suitable number of output devices 140 may be operatively connected to computing device 100. Any applicable input/output (I/O) devices may be connected to computing device 100 as shown by blocks 135 and 140. For example, a wired or wireless network interface card (NIC), a universal serial bus (USB) device or external hard drive may be included in input devices 135 and/or output devices 140.

Embodiments of the invention may include an article such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein. For example, an article may include a storage medium such as memory 120, computer-executable instructions such as executable code 125 and a controller such as controller 105. Such a non-transitory computer readable medium may be for example a memory, a disk drive, or a USB flash memory, encoding, including or storing instructions, e.g., computer-executable instructions, which when executed by a processor or controller, carry out methods disclosed herein, for example, methods of remote controlling and maintenance of devices for spreading odors. The storage medium may include, but is not limited to, any type of disk including, semiconductor devices such as read-only memories (ROMs) and/or random-access memories (RAMs), flash memories, electrically erasable programmable read-only memories (EEPROMs) or any type of media suitable for storing electronic instructions, including programmable storage devices. For example, in some embodiments, memory 120 is a non-transitory machine-readable medium.

A system according to embodiments of the invention may include components such as, but not limited to, a plurality of central processing units (CPU), GPUs, or any other suitable multi-purpose or specific processors or controllers (e.g., controllers similar to controller 105), a plurality of input units, a plurality of output units, a plurality of memory units, and a plurality of storage units. A system may additionally include other suitable hardware components and/or software components. In some embodiments, a system may include or may be, for example, a personal computer, a desktop computer, a laptop computer, a workstation, a server computer, a network device, or any other suitable computing device.

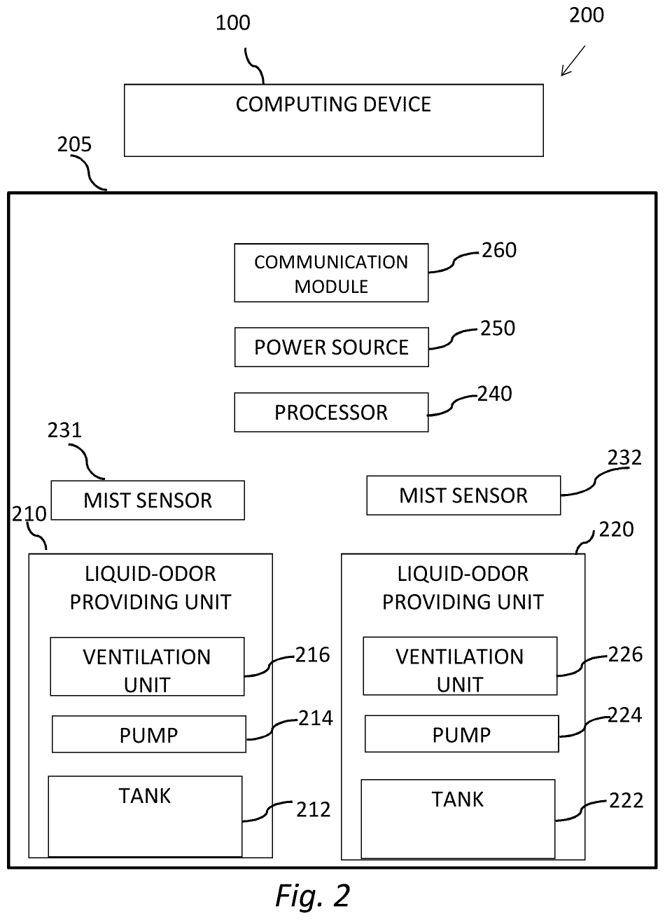

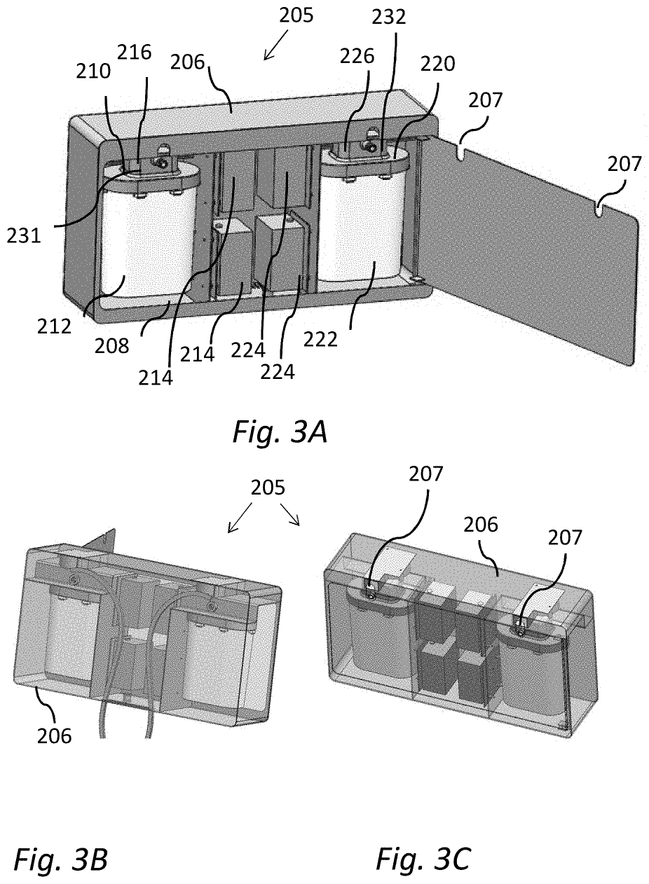

Reference is now made to FIG. 2 which is an illustration of a system 200 for remote controlling and maintenance of a device for spreading odors. System 200 may include a computing device, such as computing device (e.g., controller) 100 being in communication with one or more devices 205 for spreading odors. Each of devices 205 may include two or more liquid-odor providing units 210 and 220. In some embodiments, one or more of devices 205 may include one or more mist sensors 231 and/or 232. In some embodiments, device 205 may include a device-body 206 (illustrated and discussed in FIGS. 3A-3C) for holding two or more liquid-odor providing units 210 and 220, and one or more mist sensors 231 and/or 232. In some embodiments, mist sensors 231 and/or 232 may be any gas sensors configured to detect gases and/or aerosols spread from two or more liquid-odor providing units 210 and 220, for example, mist sensors 231 and/or 232 may be laser-based gas sensor. One or more mist sensors 231 and/or 232 may be located in proximity to the exit (e.g., exit 207 illustrated in FIGS. 3A-3C) of the odor-mist from device 205.

In some embodiments, each one of two or more liquid-odor providing units 210 and 220 may include one or more replaceable tanks (212 or 222 respectively) for holding the liquid-odor, one or more pumps (214 or 224 respectively) for pumping the liquid-odor from tank 212 or tank 222 and a ventilation unit (216 or 226 respectively). In some embodiments, replaceable tanks 212 and/or 222 may be any suitable tank or cartridge capable of holding liquid-odor, for example, replaceable tanks 212 and/or 222 may be made from at least one of: a polymer, an alloy and the like. In some embodiments, empty replaceable tanks 212 and/or 222 can be easily replaced with full ones, as illustrated in FIGS. 3A-3B. In some embodiments, liquid-odor providing unit 210 and/or 220 may include more than one pump 214 and/or 224, for example, the two pumps illustrated in FIGS. 3A-3B. In some embodiments, pumps 214 and/or 224 may be pumps working at 6-12 Volt (V) and 1-2.5 Amper (A).

In some embodiments, ventilation system 216 and/or 226 may include any device, component and/or structure that may allow spreading (e.g., diffusing) the pumped liquid-odor in an area surrounding device 205 and/or directly to the ventilation/air conditioning system of the facility. For example, ventilation system 216 and/or 226 may include at least one fan.

In some embodiments, device 205 may further include a processor 240, a power source 250 and a communication module 260. In some embodiments, processor 240 may be configured to control all the controllable components of device 205, for example, pumps 214 and/or 224, ventilation system 216 and/or 226, mist sensors 231 and/or 232 and communication module 260. In some embodiments, processor 240, power source 250 and at least some of the controllable components of device 205 may all be assembled on a printed circuit board. Processor 240 may be or may include any chip or element that is configured to execute stored instructions for operating device 205. In some embodiments, processor 240 may include or may be in communication with a memory (not illustrated) storing the instructions. For example, processor 240 may be a controller having a working voltage between 6V to 24V Direct Current (DC). Processor 240 may further include an embedded Cell module and the programming of the processor may be done via the interne, for example, by using communication module 260.

Communication module 260 may include any wired and/or wireless communication module that may allow processor 240 to send and receive information from a remote device, for example, computing device 100. For example, communication module 260 may include at least one of: Wi-Fi module, Bluetooth module, cell modems 2G/3G/CAT-M and the like.

Power source 250 may be any power source, for example, a rechargeable battery providing between 6V to 24V DC. In some embodiments, power source 250 may include a cable or a socket configured to be directly connected to the electric grid, for either providing electricity for device 205 or for recharging a rechargeable battery, for example, via a USB socket.

Reference is now made to FIGS. 3A-3C which are illustrations of device 205 for spreading odors according to some embodiments of the invention. As should be understood by one skilled in the art the design of device 205 illustrated in FIGS. 3A-3C is given as an example only and the invention is not limited to this or any other design. FIGS. 3A-3C show some of the components of device 205, according to some embodiments, as assembled inside housing 206. FIG. 3A shows a front view of device 205 when a door 209 in an opening 208 is open. FIGS. 3B and 3C are rear view and front view of the components of device 205 inside housing 206, when housing 206 is shown as having semi-transparent walls. As can be seen, replaceable tanks 212 and/or 222 may be replaced by opening, for example, door 209. Replaceable tanks 212 and/or 222 may be made or may include any suitable material, for example, polymers, metals, glass etc.

In some embodiments, housing 206 may include two or more exits 207 that may allow exit/flow of the odor-mist from device 205. In some embodiments, one or more mist-sensors 231 and/or 232 may be located in proximity to at least one exit 207, in order to detect the amount of mist coming out from (e.g., produced by) device 207. In some embodiments, device 205 may include two or more liquid-odor providing units 210 and 220. In some embodiments, each one of liquid-odor providing units 210 and 220 may include one or more (e.g., two, as illustrated) pumps 214 and 224. As should be understood by one skilled in the art a liquid-odor providing unit according to embodiments of the invention may include any number of pumps and the two pumps illustrated are given as example only. In some embodiments, each one of liquid-odor providing units 210 and 220 may include ventilation unit 216 and 226, respectively, located, for example, above tanks 212 and 222.

Device 205 may further include a PCB (not illustrated) that may include processor 240 and at least some of the controllable components of device 205 discussed in FIG. 2.

In some embodiments, computing device 100 may control both liquid-odor providing unit 210 and liquid-odor providing unit 220 to work simultaneously for providing larger amount of odor, for example, for a predetermined duration of time. In some embodiments, one or more devices 205 may include a first type of liquid-odor stored in tank 212 and second type of liquid-odor stored in tank 222. In such case, computing device 100 may control both liquid-odor providing unit 210 and liquid-odor providing unit 220 to simultaneously provide two different odors.

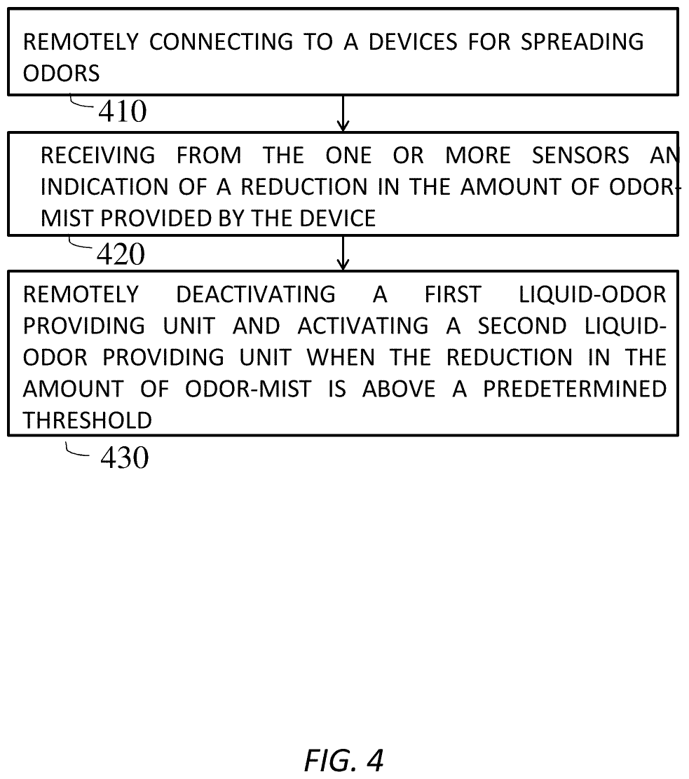

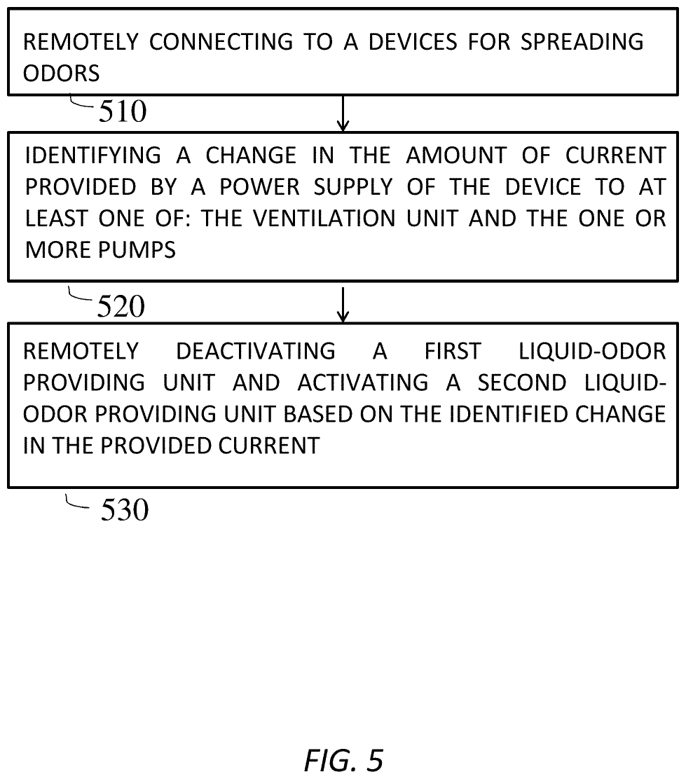

In some embodiments, system 200 may have 3 operation modes: A first operation made may include monitoring the signals received from sensors 231 and/or 232 and/or the power/current/voltage consumed by the various components of system 200. A second operation mode may include identifying/receiving a first type event, for example, regular maintenance. A third operation mode may include identifying a second type event, for example, reduction in the amount of odor mist provided and/or a change in the amount of power/current/voltage consumed by the various components of system 200. In case of the third operation mode computing device 100 may execute at least one of remote controlling and maintenance methods further described with reference to FIG. 4 and FIG. 5.

Reference is now made to FIG. 4 which is a flowchart of a method of remote controlling and maintenance of a device for spreading odors according to some embodiments of the invention. The method of FIG. 4 may be conducted by computing device 100 included in system 200 or by any other suitable computing device. In step 410, one or more devices, such as device 205, may be remotely connected, for example, via communication unit 260 to computing device 100. In some embodiments, only one liquid-odor providing unit 210 of device 205 may be operative and may provide odor-mist, while the other(s) liquid-odor providing unit(s) 220 act as reserve units.

In step 420, a signal indicative of a reduction in the amount of odor-mist provided by the device may be received from one or more mist-sensors 231 and/or 232. In some embodiments, the reduction in odor-mist may indicate a malfunction of at least one component in the operative liquid-odor providing unit 210. For example, reduction in the amount of odor-mist may occur due to: a reduction, below a threshold value, in the amount of liquid-odor in tank 211, a malfunction in one or more pumps 214 and/or a malfunction of ventilation system 216. In some embodiments, the signal may indicate a reduction due to a malfunction of sensor 231. In some embodiments, a system and method according to embodiments of the invention may detect which of the above listed problems actually occurred, and provide a solution thereto.

In step 430, when the signal indicates that the reduction in the amount of odor-mist is above a predetermined threshold, first liquid-odor providing unit 210 may be deactivated and second liquid-odor providing unit 220 may be activated. In some embodiments, deactivating first liquid-odor providing unit 210 and activating second liquid-odor providing unit 220 may be conducted by shutting down one or more pumps 214 and ventilation unit 216 and starting the operation of one or more pumps 224 and ventilation unit 226. For example, computing device 100 and/or processor 240 may control power source 250 to stop providing electric power to one or more pumps 214 and ventilation unit 216 and start providing power from power source 250 to one or more pumps 224 and ventilation unit 226. In some embodiments, computing device 100 and/or processor 240 may further deactivate mist-sensor 231 and activate mist-sensor 232.

In some embodiments, computing device 100 and/or processor 240 may further receive information related to the total operation time of device 205 since a replacement of a full tank 212 of unit 210. Computing device 100 and/or processor 240 may calculate an amount of liquid-odor consumed during the total operation time and remotely deactivating the first liquid-odor providing unit and activating the second liquid-odor providing unit. For example, computing device 100 and/or processor 240 may calculate, for example, based on known data (e.g., liquid-odor consumption per hour) stored in a database, such as, storage system 130, how much liquid-odor was consumed since the last replacement, thus may estimate the time for deactivating unit 210 and activating unit 220.

In some embodiments, computing device 100 and/or processor 240 may further be configured to send a request for a service call, each time a a deactivation/activation of the two liquid-odor providing units have being conducted. The service provided may include replacing the malfunctioned unit, such that device 100 may include, again, two functioning units. However, since a functioning unit 220 has replaced the malfunction unit 210, there is no rush in providing service to device 205, since the device is operating and providing odor-mist at the required amount. Accordingly, maintenance service for checking/repairing/replacing the malfunctioning component of unit 210 may be conducted at the convenience of the service provider.

In some embodiments, computing device 100 may further receive an indication of a change in the amount of current, voltage or power consumed by at least one of: the ventilation unit and the one or more pumps; and remotely deactivating a first liquid-odor providing unit and activating a second liquid-odor providing unit also based on the received indication. In some embodiments, controlling system 100 may include receiving/identifying the change in the amount of current and controlling the switching based solely on the identified change, as discussed with respect to the flowchart of FIG. 5.

Reference is now made to FIG. 5 which is a flowchart of a method of remote controlling and maintenance of a device for spreading odors according to some embodiments of the invention. The method of FIG. 5 may be conducted by computing device 100 included in system 200 or by any other suitable computing device. In step 510, one or more devices, such as device 205, may be remotely connected, for example, via communication unit 260 to computing device 100. In some embodiments, only one liquid-odor providing unit 210 of device 205 may be operative and may provide odor-mist, while the other(s) liquid-odor providing unit(s) 220 acts as reserve unit(s).

In step 520, a change, may be identified in the amount of current/voltage/power consumed by at least one of: the ventilation unit and the one or more pumps. In some embodiments, computing device 100 included in system 200 may identify an increase, above a first predetermined threshold value, in the amount of current/voltage/power consumed. For example, such an increase may indicate that a pipe or another component of one or more pumps 214 or 224 is blocked (e.g., plugged) therefore the pump utilizes more power (e.g., current and/or voltage) in order to overcome the block and pump the liquid from tank 212 or 222. In another example, if a filter included in tank 212 or 222 is partially or fully blocked one or more pumps 214 or 224 may utilize more power (e.g., current and/or voltage) in order to overcome the block.

In some embodiments, computing device 100 included in system 200 may identify decrease, below a second predetermined threshold value, in the amount of current/voltage/power consumed. For example, in case of wrong assembling of the tank to the pump that may cause running of air, the pump may consume less power/current/voltage since it pumps air. A similar behavior may indicate that tank 212 or 222 is empty, and one or more pumps 214 or 224 pump air.

In step 530, when change in the consumed current is identified, first liquid-odor providing unit 210 may be remotely deactivated and second liquid-odor providing unit 220 may be activated. In some embodiments, the deactivating/activating of first liquid-odor providing unit 210 and second liquid-odor providing unit 220 may be conducted by shutting down one or more pumps 214 and ventilation unit 216 and starting the operation of one or more pumps 224 and ventilation unit 226. For example, computing device 100 and/or processor 240 may control power source 250 to stop providing electric power to one or more pumps 214 and ventilation unit 216 and start providing power from power source 250 to one or more pumps 224 and ventilation unit 226.

In some embodiments, the methods of FIGS. 4 and 5 may be combined in order to have a more reliable method of detecting malfunctioning of a component of device 100. In some embodiment, when an indication of a reduction in the amount of odor-mist may be detected, in step 420, computing device 100 may further identify a change in the amount of current/voltage/power consumed by a specific component, in step 520. For example, when a reduction in the amount of odor-mist provided by unit 210 may be detected computing device 100 may identify a change in the amount of current consumed by pump 214 (e.g., an increase in the current consumption) indicating a potential blocking in the pump or one of the pipes connecting pump 214 to tank 212. In some embodiments, combining the teaching of both steps 420 and 520 may allow to detect both the malfunction of the odor providing unit and identifying which component or components included in the unit is malfunctioned.

In some embodiments, the deactivation of the first liquid-odor providing unit and the deactivation of the second liquid-odor providing unit may be conducted based on both the detected reduction in the provision of the amount of odor-mist provided and the identified change in the current/voltage/power consumed by at least one component included in the first liquid-odor providing unit.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A device for spreading odors, comprising:
one or more mist sensors; and
two or more liquid-odor providing units,
wherein each liquid-odor providing unit comprises:
one or more replaceable tanks for holding the liquid-odor;
one or more pumps for pumping the liquid-odor from the tank; and
a ventilation unit for spreading the pumped liquid-odor.

2. The device of claim 1, further comprising:
two or more mist sensors, each being associated with a different liquid-odor providing unit.

3. The device according to claim 1, wherein each mist sensor is located in proximity to an exit of the odor-mist.

4. The device of claim 1, further comprising:
a communication unit; and
a processor configured to:
receive signal indicating a reduction in the amount of odor-mist provided by the device.

5. The device of claim 4, wherein the processor is further configured to:
send the received signal to an external computing device, via the communication unit.

6. A method of remote controlling and maintenance of a device for spreading odors, comprising:
remotely connecting a device comprising:
one or more mist sensors; and
two or more liquid-odor providing units,
wherein each liquid-odor providing unit comprises:
one or more replaceable tanks for holding the liquid-odor;
one or more pumps for pumping the liquid-odor from the tank; and
a ventilation unit for spreading the pumped liquid-odor,
receiving from the one or more sensors a signal indicating a reduction in the amount of odor-mist provided by the device; and
remotely deactivating a first liquid-odor providing unit and activating a second liquid-odor providing unit when the reduction in the amount of odor-mist is above a predetermined threshold, conducted by shutting down the one or more pumps and the ventilation unit of the first liquid-odor providing unit and starting the operation of the one or more pumps and the ventilation unit of the second liquid-odor providing unit.

7. The method of claim 6, further comprising:
receiving an indication in change in the amount of current consumed by at least one of: the ventilation unit and the one or more pumps; and
remotely deactivating a first liquid-odor providing unit and activating a second liquid-odor providing unit also based on the received indication.

8. The method of claim 6, wherein each liquid-odor providing unit is associated with a different sensor and deactivating between the first liquid-odor providing unit and activating the second liquid-odor providing unit further includes shutting-down the senor of the first liquid-odor providing unit and operating the sensor of the second liquid-odor providing unit.

9. The method of claim 6, further comprising:
receiving information related to the total operation time of the device since a replacement of a first full tank;
calculating an amount of liquid-odor consumed during the total operation time; and
remotely deactivating the first liquid-odor providing unit and activating the second liquid-odor providing unit.

10. The method of claim 6, further comprising:
sending a request for a service call.

11. A system for remote controlling and maintenance of devices for spreading odors, comprising:
at least one controller; and
a communication unit configured to communicate between the at least one controller and one or more devices for spreading odors,
wherein each of the devices comprises:
one or more mist sensors; and
two or more liquid-odor providing units,
wherein each liquid-odor providing unit comprises:
one or more replaceable tanks for holding the liquid-odor;
one or more pumps for pumping the liquid-odor from the tank; and
a ventilation unit for spreading the pumped liquid-odor, and wherein the controller is configured to:
receive from the one or more sensors a signal indicating a reduction in the amount of odor-mist provided by the device; and
remotely deactivate a first liquid-odor providing unit and activate a second liquid-odor providing unit when the reduction in the amount of odor-mist is above a predetermined threshold, conducted by shutting down the one or more pumps and the ventilation unit of the first liquid-odor providing unit and starting the operation of the one or more pumps and the ventilation unit of the second liquid-odor providing unit.

12. A method of remote controlling and maintenance of a device for spreading odors, comprising:
remotely connecting a device comprising:
two or more liquid-odor providing units,
wherein each liquid-odor providing unit comprises:
one or more replaceable tanks for holding the liquid-odor;
one or more pumps for pumping the liquid-odor from the tank; and
a ventilation unit for spreading the pumped liquid-odor,
identifying a change in the amount of current consumed by at least one of: the ventilation unit and the one or more pumps; and
remotely deactivating a first liquid-odor providing unit and activating a second liquid-odor providing unit based on the identified change in the consumed current, conducted by shutting down the one or more pumps and the ventilation unit of the first liquid-odor providing unit and starting the operation of the one or more pumps and the ventilation unit of the second liquid-odor providing unit.

13. The method of claim 12, wherein identifying a change in the amount of current consumed comprises one of:
identifying an increase in the consumed current above a first predetermined threshold value; and
identifying a decrease in the consumed current below a second predetermined threshold value.
